(19) United States
(12) Patent Application Publication
(10) Pub. No.: US 2023/0285723 A1
(43) Pub. Date: Sep. 14, 2023

(54) GUIDEWIRE WITH POLYMER LAYER STRENGTHENING FEATURE

(71) Applicant: Heraeus Medical Components LLC, St. Paul, MN (US)

(72) Inventors: David JOHNSON, Fridley, MN (US); Jack DANT, Fridley, MN (US); Mark ERIE, Fridley, MN (US); Brian LONG, Fridley, MN (US); Joshua BRANDT, Fridley, MN (US)

(73) Assignee: Heraeus Medical Components LLC, St. Paul, MN (US)

(21) Appl. No.: 18/118,998

(22) Filed: Mar. 8, 2023

Related U.S. Application Data
(60) Provisional application No. 63/318,159, filed on Mar. 9, 2022.

(51) Int. Cl. A61M 25/09

(52) U.S. Cl. CPC A61M 25/09; A61M 2025/09091; A61M 2025/09158

(57) ABSTRACT

One aspect provides a medical guidewire including a core wire extending from a proximal tip of a proximal end section to a distal tip of a distal end section. A polymer layer covers a perimeter surface of at least a portion of the distal end section, the polymer layer covering the distal tip and extending proximally to a proximal end portion defining a proximal edge. A strengthening feature is disposed about a perimeter surface of at least the core wire at the proximal end portion of the polymer layer, the strengthening feature beginning at a location proximally spaced from the proximal edge of the polymer layer and extending distally at least until the proximal edge of the polymer layer, the strengthening feature to prevent damage to the proximal end portion of the polymer layer.

### CROSS-REFERENCE TO RELATED APPLICATION

This Non-Provisional patent application claims the benefit of the filing date of U.S. Provisional Patent Application Ser. No. 63/318,159, filed Mar. 9, 2022, which is incorporated herein by reference.

### TECHNICAL FIELD

Medical guidewires are commonly employed to advance intraluminal devices, such as stent delivery catheters and balloon dilatation catheters, for example, within a patient's body, such as within a patient's vasculature. Medical guidewires may also be employed to access the digestive system, urinary tract, neurology, organs for cancer treatment, etc.

### BACKGROUND

Guidewires are used in conjunction with intravascular devices, such as catheters, to facilitate navigation through the vasculature of a patient. According to some techniques, to deliver such intravascular devices to a desired location within the patient's vasculature, the guidewire is positioned within an inner lumen of the device. The guidewire is then advanced through the patient's vasculature until reaching the desired location, at which point the device is advanced over the guidewire and properly positioned at the desired location where the corresponding interventional procedure is performed (e.g., a balloon dilation procedure). Commonly, the guidewire is then withdrawn from the patient's body via the device, with the device (e.g., a catheter) remaining within the body to deliver a variety of therapies to the patient.

Medical guidewires usually comprise an elongated core member, also referred to herein as a core wire, which is typically manufactured of stainless steel or nitinol (although other materials may be employed). The core wire extends from a proximal end to a distal end, where a distal tip section of the core wire is often tapered in the distal direction to a smaller transverse dimension to increase flexibility of the distal tip section to aid in leading the tip section through tortuous paths without damaging the vascular lumen or other body lumen through which it is advanced.

To further aid in advancing the guide wire through such tortuous paths and to reduce the potential for damage to the body lumen, at least the distal tip section may be coated with a polymer layer to provide a smooth and reduced-friction outer perimeter surface and to provide the guidewire with a substantially constant outer transverse dimension. In some instances, the polymer layer may be doped with another material, such as tungsten or barium sulfate particles, for example, to increase radiopaque characteristics of the guidewire to aid in the ability of the guidewire to be seen under X-ray or fluoroscopy. However, due to interactions with mating devices, particularly when withdrawing the guidewire through a device that contains a tortuous path (e.g., sharp turns, abrupt transitions, etc.), the proximal end of the polymer coating of guidewires that have only the distal tip portion covered in polymer may be subject to damage such that polymer can become separated from the guidewire or the guidewire can become lodged in the mating device. Because there are limitations to present approaches, there is a need for the present example embodiments.

### BRIEF DESCRIPTION OF THE DRAWINGS

FIG. 1 is a longitudinal cross-sectional view generally illustrating a medical guidewire including a polymer layer strengthening feature, according to one example of the present disclosure.

FIG. 2A is a cross-sectional view generally illustrating a portion of a guidewire including a polymer layer strengthening feature comprising a heat shrink sleeve, according to one example of the present disclosure.

FIG. 2B generally illustrates a portion of a guidewire including a polymer layer strengthening feature comprising a heat shrink sleeve, according to one example of the present disclosure.

FIG. 3A is a block and schematic diagram generally illustrating a portion of a guidewire including a polymer layer strengthening feature comprising an adhesive layer, according to one example of the present disclosure.

FIG. 3B is a block and schematic diagram generally illustrating a portion of a guidewire including a polymer layer strengthening feature comprising an adhesive layer, according to one example of the present disclosure.

FIG. 3C is a block and schematic diagram generally illustrating a portion of a guidewire including a polymer layer strengthening feature comprising an adhesive layer, according to one example of the present disclosure.

FIG. 3D is a block and schematic diagram generally illustrating a portion of a guidewire including a polymer layer strengthening feature comprising an adhesive layer, according to one example of the present disclosure.

FIG. 4A is a block and schematic diagram generally illustrating a portion of a guidewire including a polymer layer strengthening feature comprising a hypotube, according to one example of the present disclosure.

FIG. 4B is a block and schematic diagram generally illustrating a portion of a guidewire including a polymer layer strengthening feature comprising a hypotube, according to one example of the present disclosure.

FIG. 5A is a block and schematic diagram generally illustrating a portion of a guidewire including a polymer layer strengthening feature comprising a proximal end section of a polymer layer formed from at least one higher durometer polymer material, according to one example of the present disclosure.

FIG. 5B is a block and schematic diagram generally illustrating a portion of a guidewire including a polymer layer strengthening feature comprising a plurality of proximal end sections of a polymer layer formed by increasingly higher durometer polymer materials, according to one example of the present disclosure.

FIG. 6A is a block and schematic diagram generally illustrating a portion of a guidewire including a polymer layer strengthening feature comprising a coil and a ramp feature, according to one example of the present disclosure.

FIG. 6B is a block and schematic diagram generally illustrating a portion of a guidewire including a polymer layer strengthening feature comprising a coil and an adhesive transition feature, according to one example of the present disclosure.

FIG. 6C is a block and schematic diagram generally illustrating a portion of a guidewire including a polymer layer strengthening feature comprising a coil and an adhesive transition feature, according to one example of the present disclosure.

### DETAILED DESCRIPTION

In the following detailed description, reference is made to the accompanying drawings which form a part hereof, and in which is shown by way of illustration specific examples in which the disclosure may be practiced. It is to be understood that other examples may be utilized and structural or logical changes may be made without departing from the scope of the present disclosure. The following detailed description, therefore, is not to be taken in a limiting sense, and the scope of the present disclosure is defined by the appended claims. It is to be understood that features of the various examples described herein may be combined, in part or whole, with each other, unless specifically noted otherwise.

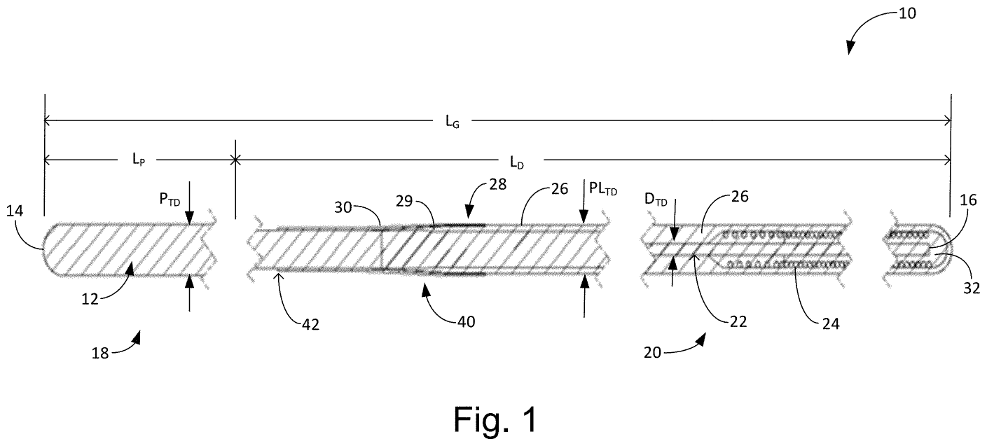

FIG. 1 is longitudinal cross-section generally illustrating a medical guidewire 10 according to one example of the present disclosure. Guidewire 10 has a proximal end section 18 and distal end section 20, and includes a core wire 12 extending from a proximal tip 14 at proximal end section 18 to a distal tip 16 at distal end section 20. In examples, core wire 12 may comprise any number suitable materials including stainless steel, Nitinol, Nitinol-based alloys, and various high-stiffness alloys, including high-performance cobalt-based alloys (e.g., 35N LT®). In one example, for a majority of an overall length, L_{G}, of guidewire 10, core wire 12 has a transverse dimension (e.g., the diameter), such as transverse dimension, P_{TD} at proximal end section 18, which is sufficient to provide good pushability properties as well as good torque qualities suitable for being advanced and rotated so as to aid in maneuvering through a patient's vascular system.

In some examples core wire 12 comprises a contiguous wire formed of a same material over its entire length. In examples, such as illustrated by FIG. 1, core wire 12 has a transverse dimension which tapers from a larger transverse dimension, P_{TD}, at proximal end section 18 of guidewire 10, to a reduced transverse dimension D_{TD}, at distal end section 20 guidewire 10. In one example, at least a distal end segment 22 of core wire 12 is tapered to provide flexibility across at least a portion of distal end section 20. The transverse cross-section of core wire 12 can have any suitable cross-sectional configuration such as circular, elliptical, triangular, square or rectangular, wherein different sections of core wire 12 may have different cross-sectional configurations.

In other examples, core wire 12 comprises different sections formed of different materials which are joined together to form core wire 12. For instance, in some examples, proximal portions of core wire 12, such as at proximal section 18, may be formed of high-stiffness materials, such as stainless steel, and distal portions of core wire 12, such as at distal end section 20, may be formed of materials exhibiting higher resiliency, including super-elastic materials such as Nitinol, for example, to provide good flexibility to distal end section 20 for traversing a patient's vasculature.

Guidewire 10 may embody a range of dimensions that are considered as appropriate for various implementations. In one example, a transverse dimension of guidewire 10, such as an outer diameter (see proximal and distal transverse dimensions P_{TD} and D_{TD} below), ranges from about 0.005 to about 0.04 inches. Additionally, guidewire 10 may be configured with a variety of lengths. In one example, an overall length, L_{G}, of guidewire 10 ranges from about 6.0 to 140.0 inches. In one example, a length, L_{D}, of distal end section 20 may range from about 0.5 to 32.0 inches. In one example, a length, L_{P}, of proximal end section 18 may range from about 5.5 to 108.0 inches. It is noted that such ranges of lengths and diameters are provided as examples, and that guidewire 10 may comprise dimensions other than the delineated ranges.

In examples, a distal helical coil 24 is disposed about and attached to distal end segment 22 using any suitable bonding technique (e.g., solder, adhesive). In examples, distal helical coil 24 is formed of a radiopaque material, such as platinum, or is formed of other materials, such as stainless steel, and coated with a radiopaque material, such as gold, for example. In examples, a pitch of distal helical coil 24 may change over its length (e.g., “stretched” at its proximal end) to increase flexibility and/or to aid in application of glue, solder, etc.

In one example, a polymer layer 26 is disposed about a perimeter of at least a portion of distal end section 20, wherein polymer layer 26 covers distal tip 16 and extends proximally to a proximal end portion 28 defining a proximal edge 30. In one example, distal helical coil 24 is embedded within polymer layer 26. In one example, distal helical coil 24 is embedded within an additional polymer layer 32, with polymer layer 26 disposed about a perimeter surface of the additional polymer layer 32. Polymer layer 26 may be of any suitable polymer material including polyethylene terephthalate (PTE), Pellethane®, Tecoethane™, etc. Polymer layer 26 may be applied using any suitable method including heat shrinking, dipping, spraying, painting, vapor deposition, and molding, for example, which produces a smooth and continuous outer surface.

In one example, polymer layer 26 may be applied in the form of a polymer preform which is disposed about core wire 12, with a heat shrink sleeve disposed about both the polymer preform and core wire 12. Upon application of heat, the polymer preform reflows while the heat shrink sleeve contracts to compress the reflowed polymer about core wire 12. The heat shrink sleeve is then removed, leaving polymer layer 26 with a smooth and continuous outer surface.

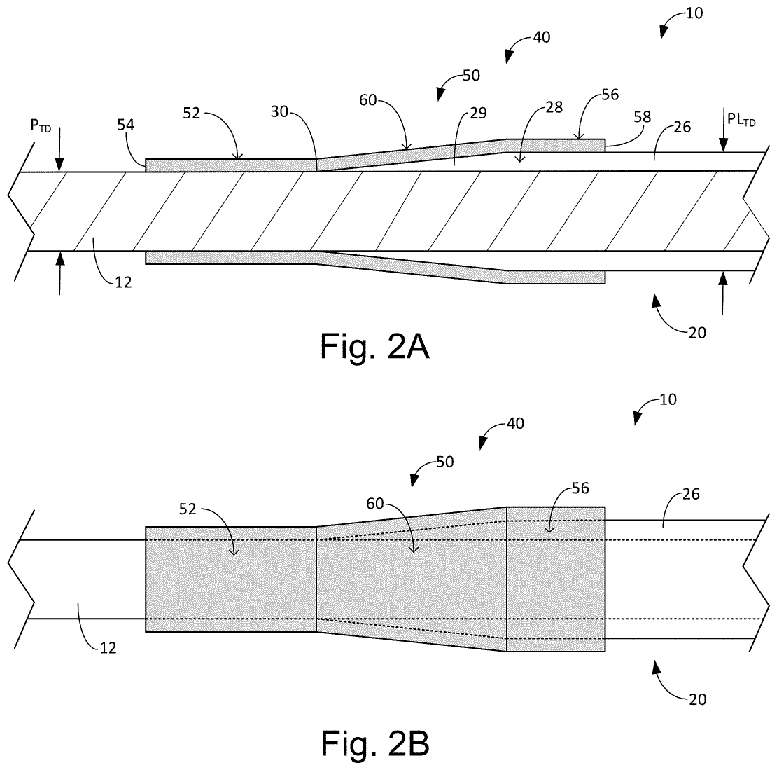

In some examples, polymer layer 26 has an outer transverse dimension, PL_{TD}, similar to outer transverse dimension, P_{TD}, of core wire 12 at proximal end section 18 so as to provide guidewire 10 with a substantially constant outer transverse dimension which translates smoothly in an axial direction within catheter lumens, intracorporeal channels, and the like. In other examples, the outer transverse dimension, PL_{TD}, of polymer layer 26 may be variable along an axial length thereof to produce a tapered outer transverse dimension, where such a tapered outer dimension can be tapered distally and/or proximally, for example, and which “follows” a tapered shape of core wire 12, a shape of a coil, etc., resulting in an outer transverse dimension that is not uniform. In one example, as illustrated in FIG. 1 (and as will be illustrated in greater detail below, such as by FIGS. 2A and 2B), proximal end portion 28 of polymer layer 26 is tapered in the proximal direction (i.e., toward proximal tip 14) to form a ramp-like portion 29 that transitions from outer transverse dimension, PL_{TD}, of polymer layer 26 to the outer transverse dimension, P_{TD}, of core wire 12 at proximal edge 30. In one example, ramp-like portion 29 is produced as part of the heat shrink forming process of polymer layer 26, as described above, where, when heated, a proximal portion of the reflowed polymer material spreads below the heat shrink sleeve to form ramp-like portion 29. In other examples, ramp-like portion 29 may be formed after formation of polymer layer 26, such as via grinding or machining techniques, for example.

While polymer layer 26 provides a soft, smooth surface over core wire 12 to provide increased ease of insertion into a patient's vascular system and protection from vascular perforation, when withdrawing guidewire 10, such as through a tortuous path of a mating device and/or when contacting sharp or hard openings of a mating device (e.g., Sheaths, Catheters, Microcatheters, and Introducers), polymer layer 26, particularly at proximal end portion 28 and proximal edge 30, may become damaged. Such damage may include portions of polymer layer 26 becoming separated from guidewire 10, where separated portions of polymer layer 26 may cause the guidewire to become lodged in the mating device and can represent a danger to a patient.

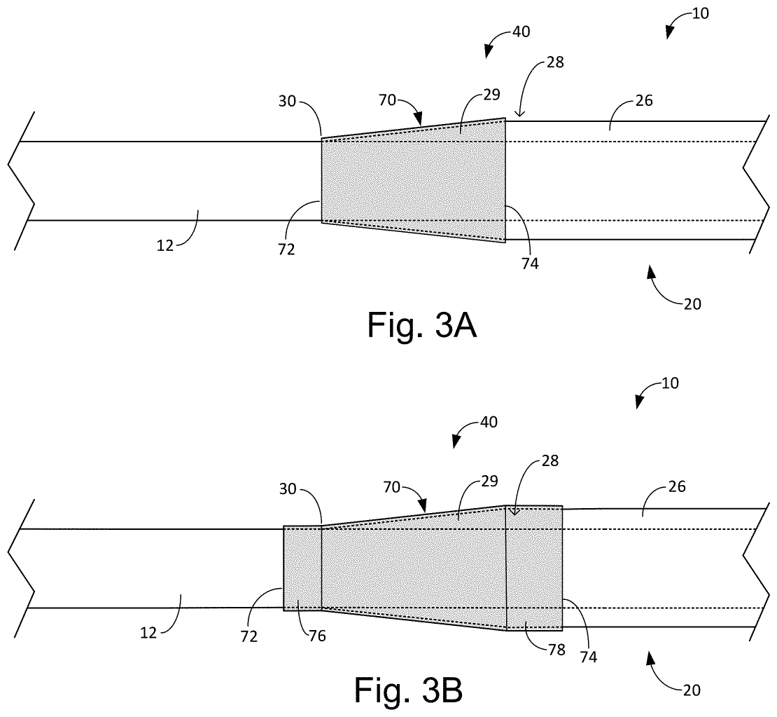

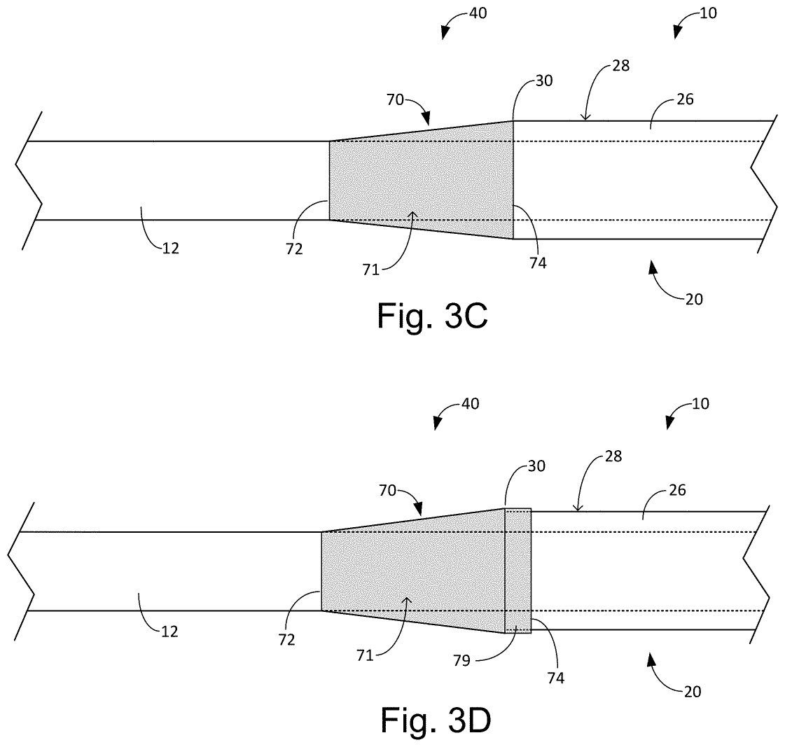

In accordance with the present application, guidewire 10 includes a strengthening feature 40 disposed along distal end section 20 of core wire 12 at proximal end portion 28 of polymer layer 26. In one example, as illustrated by FIG. 1, strengthening feature 40 has a proximal end 42 beginning at a location spaced proximally from the proximal edge 30 of polymer layer 26 and extending distally until at least the proximal edge 30 of polymer layer 26 (and, according to some examples, extending distally beyond the proximal edge 30 of polymer layer 26), wherein the strengthening feature strengthens and prevents damage (e.g., fraying, separation) to the proximal end portion 28 of the polymer layer 26, particularly along proximal edge 30 thereof. In one example, as illustrated in FIG. 1, and as illustrated in greater detail below by FIGS. 2A and 2B, strengthening feature 40 comprises a heat shrink sleeve 50 disposed about a perimeter surface of at least proximal end portion 28 of polymer layer 26. In other examples, as described in greater detail by FIGS. 3A to 6C, strengthening feature comprises shaped adhesive, hypotubes, coils, sections of higher durometer polymers, and/or combinations thereof.

FIG. 2A is an enlarged cross-sectional view, and FIG. 2B is an enlarged plan view of the proximal portion of the distal end section 20 of guidewire 10, wherein strengthening feature 40 comprises a heat shrink sleeve 50, according to one example of the present disclosure. In one example, as illustrated, proximal end portion 28 of polymer layer 26 is tapered in the proximal direction to form ramp-like portion 29 that transitions from outer transverse dimension, PL_{TD}, of polymer layer 26 to the outer transverse dimension, P_{TD}, of core wire 12 at proximal edge 30 of polymer layer 26. In examples, heat shrink layer 50 is applied after formation of polymer layer 26, wherein it is noted that in a scenario where a removable heat shrinking is employed to form polymer layer 26, heat shrink layer 50 is separate from and applied after removal of such removable heat shrinking.

In examples, heat shrink layer 50 is applied at least about the perimeter surface of ramp-like portion 29 of proximal end portion 28 of polymer layer 26. In one example, as illustrated, heat shrink sleeve 50 includes a proximal portion 52, a distal portion 56, and a central portion 60. Proximal portion 52 defines a proximal edge 54 and covers a portion of the perimeter surface of core wire 12 extending proximally from proximal edge 30 of polymer layer 26. Distal portion 56 defines a distal edge 58 and covers a portion of the perimeter surface of proximal end portion 28 of polymer layer 26 extending distally from ramp-like portion 29. Central portion 60 extends between proximal portion 52 and distal portion 56 and covers ramp-like portion 29 of proximal end portion 28 of polymer layer 26.

Although not illustrated, in some examples, heat shrink sleeve 50 includes only central portion 60 covering only ramp-like portion 29 of proximal end portion 28 of polymer layer 26. In some examples, heat shrink sleeve includes only proximal portion 52 and central portion 60. In other examples, heat shrink sleeve includes only central portion 60 and distal portion 56.

In one example, heat shrink sleeve 50 comprises a polymer material having a higher durometer than the polymer material of polymer layer 26 so as to provide improved strength to prevent potential damage to proximal end portion 28 of polymer layer 26, such as may otherwise potentially occur when advancing and/or withdrawing guidewire 10 along a tortuous path. In one example, heat shrink sleeve 50 is made of a PET material.

FIG. 3A is an enlarged view of the proximal portion of the distal end section 20 of guidewire 10, generally illustrating strengthening feature 40 as comprising an adhesive layer 70 covering at least a portion of proximal end portion 28 of polymer layer 26, according to one example of the present disclosure. In one example, as illustrated by FIG. 3A, after formation of polymer layer 26, where proximal end portion 28 of polymer layer 26 is shaped to include ramp-like portion 29, adhesive layer 70 is applied to cover at least ramp-like portion 29 such that a proximal edge 72 of adhesive layer 70 coincides with proximal edge 30 of polymer layer 26, and a distal edge 74 of adhesive layer 70 coincides with a distal edge of ramp-like portion 29.

Adhesive layer 70 may comprise any suitable type of biocompatible adhesive such as UV cured, blue light cured, or heat cured cyanoacrylates, for example. In examples, adhesive layer 70 serves as a protective layer over and strengthens proximal end 28 of polymer layer 26 to prevent potential damage to proximal end portion 28 of polymer layer 26, such as may otherwise potentially occur when advancing and/or withdrawing guidewire 10 along a tortuous path.

In other examples, as illustrated by FIG. 3B, in addition to being applied to cover ramp-like portion 29 of proximal end portion 28 of polymer layer 26, adhesive layer 70 includes proximal portion 76 applied over a portion of core wire 12 extending proximally from proximal edge 30 of polymer layer 26 (such that proximal edge 72 of adhesive layer 70 is spaced proximally from proximal edge 30 of polymer layer 26) and/or includes a distal portion applied over a region of end portion 28 of polymer layer 26 extending distally from ramp-like portion 29.

As illustrated by FIG. 3C, according to one example, where proximal edge 30 of proximal end portion 28 of polymer layer 26 comprises a blunt edge rather than being shaped to include a ramp-like portion 29, adhesive layer 70 is applied so as form a ramp structure 71 disposed proximally to proximal edge 30 so as to form a sloped transition from the outer perimeter surface of core wire 12 to the outer perimeter surface of polymer layer 26. As illustrated, in one example, proximal edge 72 of adhesive layer 70 is spaced proximally from proximal edge 30 of polymer layer 26 and distal edge 74 is coincident with proximal edge 30. As with the examples adhesive layers 70 described by FIGS. 3A and 3B, adhesive layer 70 is applied after formation of polymer layer 26. In one example, as illustrated by FIG. 3D, ramp structure 71 of adhesive layer 70 is formed with a portion 79 extending distally beyond proximal edge 30 of polymer layer 26.

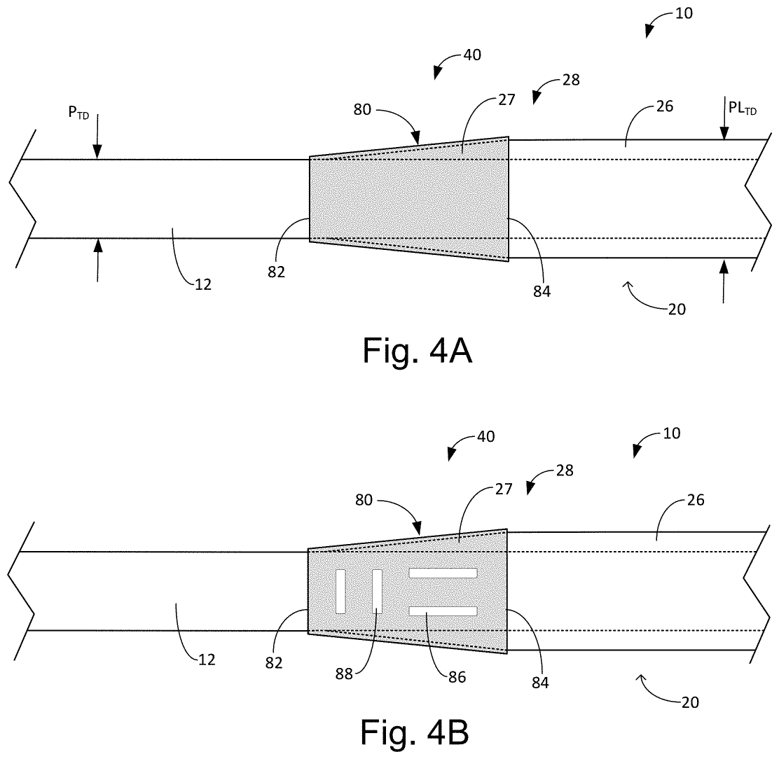

FIG. 4A is an enlarged view of the proximal portion of the distal end section 20 of guidewire 10, generally illustrating strengthening feature 40 as comprising a hypotube 80 disposed at proximal end portion 28 of polymer layer 26, according to one example of the present disclosure. Hypotube 80 extends between a proximal end 82 and a distal end 84. In one example, during formation of guidewire 10, hypotube 80 is attached to core wire 12 using any suitable attachment process (e.g., solder, adhesive, laser welding, swaging, crimping, etc.). In examples, hypotube 80 may be attached to core wire 12 at one or more points along a length of hypotube 80, or may be attached to core wire 12 along its entire length. Hypotube 80 may comprise materials such as stainless steel, gold-tungsten, tungsten, Nitinol, Inconel, or any material compatible with the material of core wire 12.

In one example, which is not illustrated, distal end 84 abuts proximal edge 30 of polymer layer 26. In other examples, such as illustrated by FIGS. 4A and 4B, a portion 27 of polymer layer 26 extends within an interior of hypotube 80. In one example, during formation of guidewire 10, polymer layer 26 is reflowed into distal end 84 so as to be disposed within the interior of hypotube 80. In other examples, which are not illustrated, polymer layer 26 may be reflowed so as to be disposed within the interior of hypotube 80 and over a portion of an exterior surface of hypotube 80 proximal to distal end 84.

In examples, such as illustrated by FIGS. 4A and 4B, hypotube 80 has tapered or cone shape with its transverse dimension (e.g., a perimeter) increasing from proximal end 82 to distal end 84 so as to form a ramp-like profile to transition from outer transverse dimension, P_{TD}, of core wire 12 to outer transverse dimension, PL_{TD}, of polymer layer 26. In other examples, hypotube 80 may be cylindrical in shape rather than cone-shaped.

In one example, as illustrated by FIG. 4B, hypotube 80 includes a number of longitudinally extending openings 86 and/or radially openings 88 extending there through. In examples, during a reflow of polymer layer 26, reflowed polymer material of polymer layer 26 flows into openings 86 and/or 88 and interlocks proximal end 28 of polymer layer 26 with hypotube 80. In examples, reflowed polymer material of polymer layer 26 disposed within opening 86 and/or opening 88 is flush with an exterior perimeter surface of hypotube 80.

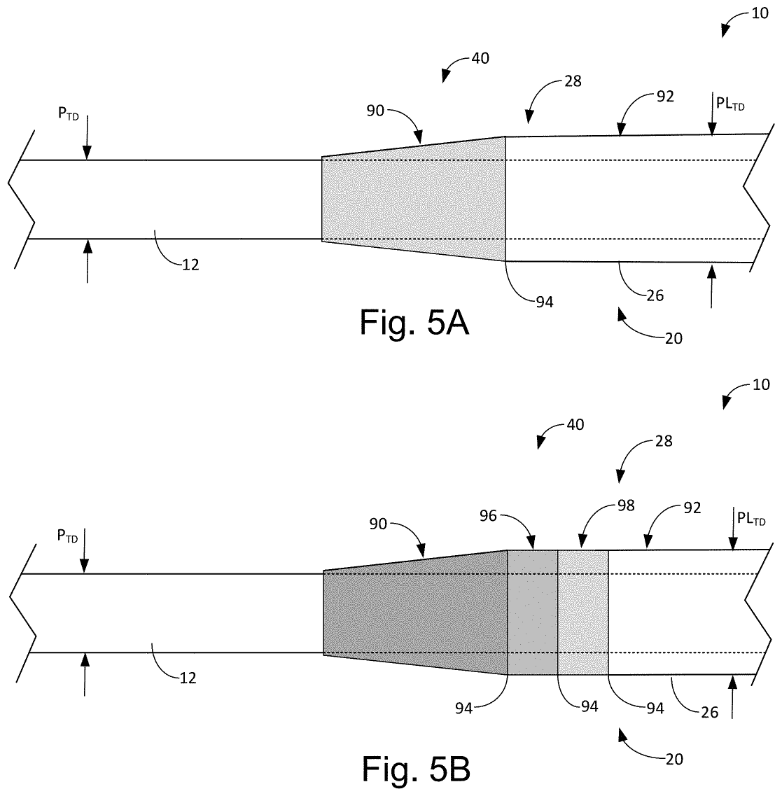

FIG. 5A is an enlarged view of the proximal portion of the distal end section 20 of guidewire 10, generally illustrating strengthening feature 40 as comprising a proximal end section 90 of polymer layer 26 where, according to one example of the present disclosure, proximal end section 90 comprises an integral portion of polymer layer 26 and is formed by a first polymer material that has a higher durometer than a primary polymer material forming a remaining distally extending portion 92 of polymer layer 26. In one example, the first polymer material of end section 90 may have a durometer value of 55D, while the primary polymer material of the remaining distally extending portion 92 of polymer layer 26 may have a durometer value of 85 A, for example. It is noted that such durometer values are provided for illustrative examples, and that any number of suitable durometer values may be employed. Forming proximal end portion 28 of polymer layer 26 with a higher durometer polymer material reduces the potential for damage to proximal end portion 28 of polymer layer 26 while providing a smooth and continuous surface to aid in movement of guidewire 10 along tortuous paths.

According to one example, during formation of guidewire 10, a first polymer preform of the first polymer material to form proximal end section 90 is placed about core wire 12, and a primary polymer preform of the primary polymer material to form the remaining distally extending portion 92 of polymer layer 26 is placed around core wire 12 and abutting the first polymer preform. A heat shrink sleeve is placed about the first polymer preform and the primary polymer perform, heat is applied to reflow the first polymer preform and the primary polymer preform and to contract the heat shrink sleeve to compress the reflowed first and primary polymer preforms, which blend together to form a seamless junction region 94 there between. The heat shrink sleeve is then removed leaving contiguous polymer layer 26 with a smooth and continuous outer surface. In one example, as illustrated, proximal end section 90 is tapered or conical in shape to form a ramp-like profile to transition from outer transverse dimension, P_{TD}, of core wire 12 to outer transverse dimension, PL_{TD}, of polymer layer 26. In some examples, such tapering may be achieved during formation of polymer layer 26 (e.g., via a heat shrinking process, as described above) or after formation of polymer layer 26 (e.g., via a machining process). In other examples, proximal end section 90 may be cylindrical in shape.

FIG. 5B is an enlarged view of the proximal portion of the distal end section 20 of guidewire 10 where strengthening feature 40 comprises multiple integrated proximal end sections of polymer layer 26, illustrated as proximal end sections 90, 96 and 98. In one example, the polymer material of each of the proximal end sections 90, 96 and 98 has a durometer greater than the durometer of the primary polymer material of the remaining distally extending portion 92 of polymer layer 26, with the polymer material of each of the proximal end sections sequentially increasing in durometer in the proximal direction (i.e., durometer of proximal end section 90>durometer of proximal end section 96>durometer of proximal end section 98>durometer of remaining distally extending portion 92 of polymer layer 26). In one example, similar to that described above with respect to FIG. 5B, each of the end sections 90, 96, and 98, and remaining portion 92 of polymer material comprise separate polymer preforms which seamlessly blend together when reflowed to form a seamless junction region 94 of corresponding blended polymer materials is between each proximal section of polymer layer 26.

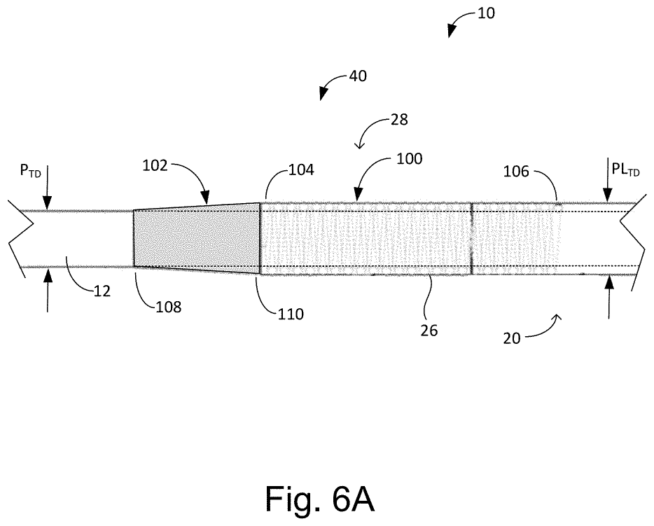

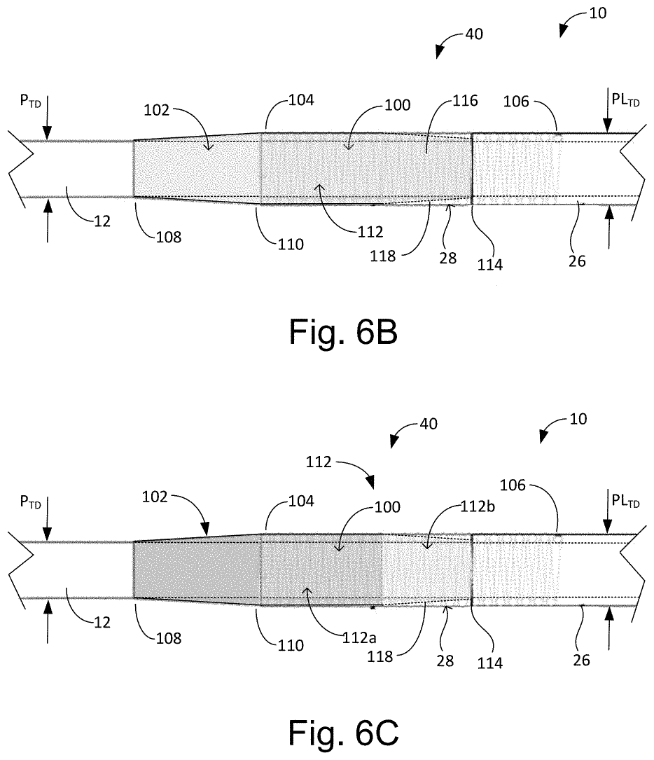

FIG. 6A is an enlarged view of the proximal portion of the distal end section 20 of guidewire 10, generally illustrating strengthening feature 40 as comprising a coil 100 and a ramp feature 102 disposed at proximal end portion 28 of polymer layer 26, according to one example of the present disclosure. Coil 100 is disposed helically about and attached to the outer perimeter surface of core wire 12, and has a proximal end 104 and a distal end 106. Coil 100 may be constructed of stainless steel, gold-tungsten, tungsten, nitinol, Inconel, or any material compatible with the material of core wire 12, and may be attached to core wire 12 using any suitable technique such as via laser welding, soldering, swaging, crimping, or adhesive, for example. In examples, coil 100 may be attached to core wire 12 at one or more points along its length or along its entire length.

In examples, coil 100 may be constructed of a variety of wire cross-sections, such as round, oval, or rectangular flat wire, for example. Additionally, coil 100 may have different diameters and pitch depending on the diameter of core wire 12. In examples, coil 100 may have a constant, variable or tapering outer diameter over its length from proximal end 104 to distal end 106.

In one example, as illustrated, an entire length of coil 100, from distal end 106 to proximal end 104, is embedded within polymer layer 26. In other examples, which are not illustrated, coil 100 may be disposed proximally from proximal end 30 of polymer layer 26 and positioned along core wire 12 such that distal end 106 abuts proximal end 30 of polymer layer 26, or only a portion of the length of coil 100 may be embedded within polymer layer 26 beginning at distal end 106. In one example, coil 100 may be attached to core wire 12 after formation of polymer layer 26 has been completed such that distal end 106 abuts proximal edge 30 of polymer layer 30. In other examples, during formation of guidewire 10, coil 100 is attached to core wire 12, after which a polymer pre-form sleeve for forming polymer layer 26 is disposed about distal end section 20 of core wire 12. In one example, the polymer preform is disposed so as to abut distal end 106 of coil 100. In one example, the polymer preform is disposed so as to extend over a portion of the length of coil 100 beginning at distal end 106. In one example, the polymer preform is disposed over an entire length of coil 100. A heat shrink sleeve is then placed about the polymer preform and coil 100, and heat is applied to reflow the polymer preform and to contract the heat shrink sleeve to compress the reflowed polymer material at least about core wire 12. In examples, the reflowed polymer material may flow so as to abut distal end 106 of coil 100, flow into a portion of coil 100 beginning at distal end 106, or flow into and across an entire length of coil 100 to form polymer layer 26.

Ramp feature 102 has a proximal end 108 and a distal end 110. In one example, ramp feature is positioned about core wire 12 such that distal end 110 of ramp feature 102 abuts proximal end 104 of coil 100. In examples, after attachment of coil 100 to core wire 12 and formation of polymer layer 26, ramp feature 102 is disposed about core wire 12 at proximal end 104 of coil 100. In one example, ramp feature 102 is formed and shaped using a solder material. In one example, ramp feature 102 may comprise any suitable type of biocompatible adhesive such as UV cured, blue light cured, or heat cured cyanoacrylates, for example. In examples, a transverse dimension of ramp feature 102 increases from proximal end 108 to distal end 110 to form a smooth transition from outer transverse dimension, P_{TD}, of core wire 12 to outer transverse dimension, PL_{TD}, of polymer layer 26 (and coil 100).

Positioning coil 100 at the proximal end of polymer layer 26 protects at least proximal edge 30 of polymer layer 26 from potential damage when advancing or retracting guidewire 10 along a tortuous path, such as through a mating device or a patient's vascular system. Reflowing proximal end portion 28 of polymer layer 26, including proximal edge 30, either partially or fully into coil 100, affixes and interlocks polymer layer 26 with coil 100 such that proximal end portion 28 of polymer layer 26, and particularly proximal edge 30, are more resistant to damage and peeling. Positioning ramp element 102 at proximal end 104 of coil 100 provides a smooth transition between core wire 12 and coil 100 so as to ease movement of guide wire 10 along a tortuous path, thereby further reducing the potential occurrence of damage to polymer layer 26.

FIG. 6B is an enlarged view of the proximal portion of distal end section 20 of guidewire 10 illustrating an example implementation of strengthening feature 40 where, in addition to an adhesive material forming ramp feature 102 as illustrated by FIG. 6A, where ramp feature 102 ends at and abuts proximal end 104 of coil 100, the adhesive material of strengthening feature 40 further includes an extension feature 112 which extends distally beyond ramp feature 102 and over/into at least a portion of coil 100. In some examples, as illustrated, extension feature 112 of adhesive material extends from ramp feature 102 to a distal edge 114 which is short of distal end 106 of coil 100. In some examples (not illustrated), extension feature 112 may extend over a full length of coil 100 such that distal edge 114 is coincident with distal end 106 of coil 100. In other examples (not illustrated), extension feature 112 may extend distally beyond the distal end 106 of coil 100. In one example, as illustrated by FIG. 6B, ramp feature 102 and extension feature 112 of strengthening feature 40 are of a same adhesive material with extension feature 112 extending seamlessly from ramp feature 102.

In one example, as illustrated, a distal end portion 116 of extension feature 112 is tapered, as indicated at 118, such that a transverse dimension (e.g., a diameter) of extension feature 112 at distal edge 114 is less than a transverse dimension at distal edge 110 of ramp feature 102. In one example, as illustrated, proximal end portion 28 of polymer layer 26 is disposed over and overlaps with the tapered distal end portion 116 of extension feature 112. In other examples (not illustrated), distal end portion 116 of extension feature 112 may not be tapered, where proximal end portion 28 of polymer layer is disposed over the overlaps with such non-tapered distal end portion of extension feature 112.

FIG. 6C is an enlarged view of the proximal portion of distal end section 20 of guidewire 10 illustrating an example implementation of strengthening feature 40 where, in a fashion similar to that described above by FIG. 5B (with respect to polymer layer 26), extension feature 112 includes multiple sections or sub-portions formed by adhesive materials having different durometers. According to examples, a durometer of ramp feature 102 and each successive sub-portion of extension feature 112 increases in the proximal direction (or conversely, decreases in the distal direction). In one example, as illustrated, strengthening feature 40 includes ramp feature 102 and extension feature 112, with extension feature 112 further including sub-portions 112a and 112b.

In examples, ramp feature 102, sub-portion 112a, and sub-portion 112b are each formed of an adhesive material having a different durometer, where the durometers of the adhesive material increases in the proximal direction such that the durometer of ramp feature 102 is greater than the durometer of sub-portion 112a, and the durometer of sub-portion 112a is greater than the durometer of sub-portion 112b, and the durometer of sub-portion 112b of strengthening feature 40 is greater than the durometer of polymer layer 26. In examples, the transition between each of the different portions of strengthening element 40 and between strengthening feature 40 and polymer layer 26 is formed so as to be seamless (e.g., via a reflow and heat shrink process as described above). Extending the adhesive material of strengthening feature 40 over/into coil 100 affixes and interlocks the adhesive material with coil 100, and strengthens a transition from strengthening feature 40 to polymer layer 26 to thereby further reduce the potential occurrence of damage to polymer layer 26, particularly at its proximal edge 30.

Although specific examples have been illustrated and described herein, a variety of alternate and/or equivalent implementations may be substituted for the specific examples shown and described without departing from the scope of the present disclosure. This application is intended to cover any adaptations or variations of the specific examples discussed herein. Therefore, it is intended that this disclosure be limited only by the claims and the equivalents thereof.

1. A medical guidewire comprising:
a core wire extending from a proximal tip of a proximal end section to a distal tip of a distal end section;
a polymer layer covering a perimeter surface of at least a portion of the distal end section, the polymer layer covering the distal tip and extending proximally to a proximal end portion defining a proximal edge; and
a strengthening feature disposed about a perimeter surface of at least the core wire at the proximal end portion of the polymer layer, the strengthening feature beginning at a location proximally spaced from the proximal edge of the polymer layer and extending distally at least until the proximal edge of the polymer layer, the strengthening feature to prevent damage to the proximal end portion of the polymer layer.

2. The medical guidewire of claim 1, the strengthening feature comprising:
a coil disposed helically about the perimeter surface of the core wire at the proximal end of the polymer layer, at least a portion of the coil coupled to the core wire between a proximal end and a distal end of the coil; and
a ramp element disposed about the perimeter surface of the core wire and having a proximal end and a distal end, the proximal end of the ramp element spaced proximally from the proximal end of the coil and the distal end of the ramp element abutting the proximal end of the coil, wherein a diameter of the ramp element increases from the proximal end to the distal end so as to form a sloped surface to transition from the perimeter surface of the core wire to an outer perimeter of the coil, the ramp element comprising one of an adhesive material and a solder material.

3. The medical guidewire of claim 1, wherein the distal end of the coil abuts the proximal end of the polymer layer or wherein, beginning at the distal end of the coil, a portion of the coil embedded within the polymer layer such that the proximal end of the first polymer layer is disposed between the distal and proximal ends of the coil.

4. The medical guidewire of claim 1, wherein the entire length of the coil, from the distal end to the proximal end, is embedded within the first polymer layer, with the proximal end of the polymer layer being coincident with the proximal end of the coil.

5. The medical guidewire of claim 1, wherein a diameter of the coil increases from the proximal end to the distal end of the coil or wherein a pitch the coil changes from the proximal end to the distal end of the coil.

6. The medical guidewire of claim 1, wherein the strengthening feature comprises a hypotube having a proximal end and a distal end, the hypotube attached to the core wire such that the proximal end is spaced from the proximal end of the polymer layer and the distal end at least abuts the proximal end of the polymer layer, wherein an exterior surface of the hypotube at the distal end is substantially flush with an exterior surface of the polymer layer.

7. The medical guidewire of claim 6, wherein the proximal end of the polymer layer is disposed within an interior space of the hypotube.

8. The medical guidewire of claim 6, wherein the hypotube include a plurality of openings extending through the sidewall, wherein the polymer layer is disposed within the openings to interlock the polymer layer with the hypotube or wherein the hypotube is tapered so as to be narrower at the proximal end than at the distal end.

9. The medical guidewire of claim 1, wherein the proximal end portion of the polymer layer is tapered to form a ramp to transition from an exterior surface of the core wire to an exterior surface of the polymer layer, and wherein the strengthening feature comprises a heat shrink layer disposed over at least the ramp formed by the proximal end portion of the polymer layer.

10. The medical guidewire of claim 9, wherein the heat shrink layer is disposed over the ramp and over a portion of the core wire extending proximally from the proximal end of the polymer layer or wherein the heat shrink layer is further disposed over a portion of the polymer layer extending distally from the ramp.

11. The medical guidewire of claim 1, wherein the strengthening feature comprises an adhesive ramp element disposed about the perimeter surface of at least the core wire and having a proximal end and a distal end, the proximal end of the ramp element spaced proximally from the proximal edge of the polymer layer and the distal end of the adhesive ramp element extends distally at least to the proximal edge of the polymer layer, wherein a diameter of the adhesive ramp element increases from the distal end to the proximal end so as to form a sloped surface to transition from the perimeter surface of the core wire to an outer perimeter of the coil.

12. The medical guidewire of claim 11, wherein the distal end of the adhesive ramp element overlaps with the proximal end portion of the polymer layer.

13. The medical guidewire of claim 11, wherein a proximal end portion of the polymer layer is tapered in the proximal direction with the adhesive material of the adhesive ramp covering the tapered proximal end portion of the polymer layer.

14. The medical guidewire of claim 1, wherein the strengthening feature comprises at least one proximal end section of the polymer layer being formed by a first polymer material that is different from a primary polymer material forming a remaining portion of the polymer layer, wherein the first polymer material has a durometer greater than the primary polymer material, wherein the first polymer material and the primary polymer material together form the polymer layer, and wherein the first polymer material of the at least one proximal end section and the primary polymer material of the remaining portion of the polymer layer blend together in a seamless junction region.

15. The medical guidewire of claim 14, wherein the strengthening feature comprises a plurality of proximal end sections of the polymer layer, where each proximal end section of the plurality of proximal end sections comprises a polymer material different from one another and from the primary polymer material, where each of the plurality of additional end sections are sequentially disposed in the proximal direction from the primary polymer material in an order of increasing durometer such that the polymer material of the proximal end section at the proximal end of the polymer layer has the highest durometer.

16. A medical guidewire comprising:
a proximal end section;
a distal end section;
a core wire extending from a proximal tip at the proximal end section to a distal tip of at the distal end section;
a polymer layer covering a perimeter surface of at least a portion of the distal end section, the polymer layer covering the distal tip core wire and extending proximally to a proximal end portion of the polymer layer which defines a proximal edge of the polymer layer; and
a strengthening feature disposed at the proximal end portion of the polymer layer, the strengthening feature including:
a coil helically disposed about a perimeter surface of the core wire and coupled thereto; and
an adhesive material layer disposed about a perimeter surface of the core wire, the adhesive material layer including:
a ramp feature having a proximal edge spaced proximally from a proximal end of the coil and an extending to a distal edge abutting the proximal end of the coil, the ramp feature having a transverse dimension which increases from the proximal edge to the distal edge to form a sloped surface to transition from the perimeter surface of the core wire to an outer perimeter of the coil; and
an extension feature that extends distally from the distal edge of the ramp feature to cover at least a portion of the coil, wherein the polymer layer extends proximally to cover and overlap a distal portion of the extension feature.

17. The medical guidewire of claim 16, wherein the extension feature covers a first portion of the coil and the polymer layer covers a remaining portion of the coil or wherein the extension feature extends distally to a distal end of the coil.

18. The medical guidewire of claim 16, wherein the ramp feature and the extension feature of adhesive material layer are of a same material.

19. The medical guidewire of claim 16, wherein at least a portion the extension feature of the adhesive material layer has a transverse dimension which matches a transverse dimension of the ramp feature at the proximal end of the coil or wherein a distal portion of the extension feature of the adhesive material layer has a tapered transverse dimension wherein a distal edge of the extension feature of the adhesive layer is smaller than a transverse dimension of a distal edge of the ramp feature.

20. The medical guidewire of claim 16, wherein the extension feature of the adhesive material layer comprises a plurality of sub-portions, wherein the sub-portions and the ramp feature each comprise an adhesive material having a different durometer, wherein each successive sub-portion has a decreasing durometer in the distal direction.
